(19) United States
(12) Patent Application Publication
(10) Pub. No.: US 2018/0008164 A1
(43) Pub. Date: Jan. 11, 2018

(54) NON-INVASIVE METHOD AND SYSTEM FOR ASSESSING SURVIVAL OF TRANSPLANTED FLAP

(71) Applicant: CHIMEI MEDICAL CENTER, Tainan City (TW)

(72) Inventors: JHI-JOUNG WANG, TAINAN CITY (TW); BOR-SHYH LIN, TAINAN CITY (TW); CHUN-CHIA CHEN, TAINAN CITY (TW)

(21) Appl. No.: 15/206,403

(22) Filed: Jul. 11, 2016

(51) Int. Cl. A61B 5/053; A61B 5/00; A61F 2/10; A61B 17/00

(52) U.S. Cl. CPC A61B 5/0531; A61F 2/105; A61B 5/7282; A61B 5/4848; A61B 5/7225; A61B 5/4851; A61B 2017/00969

(57) ABSTRACT

A non-invasive method and system for assessing the survival of a transplanted flap involve the following steps. A control unit instructs a variable-frequency current generator circuit to generate a constant current at a fixed frequency and pass the constant current through a detection electrode. The detection electrode detects a bioelectrical impedance of the skin to which the flap has been transplanted, and the bioelectrical impedance of the skin is compared with a pre-defined threshold value. If the bioelectrical impedance of the skin exceeds the pre-defined threshold value, it is determined that an abnormal condition has occurred.

### BACKGROUND OF THE INVENTION

### 1. Technical Field

The present invention relates to a non-invasive method and system for assessing the survival of a transplanted flap. More particularly, the invention relates to a method and system for assessing the survival of a flap by detecting with a detection electrode the bioelectrical impedance of the skin to which the flap has been transplanted.

### 2. Description of Related Art

As disclosed in http://sunifeng.blogspot.tw/2015/08/blog-post_94.html, the most important issue after a flap surgery is to ensure that the flap in question survives, and whether a flap survives or not can be determined by observation, in particular by inspection, by palpation, by stabbing with a needle, and with a thermometer.

In other words, the survival of a transplanted flap can be determined by: 1) the color of the flap; 2) the flap's reaction to a capillary blanching test; 3) the warmth of the flap; and 4) how a stab wound of the flap bleeds.

The characteristics of a failed flap include: 1) a continuous decrease in temperature (a rapid decrease can be found where an artery lies, and a slow decrease can be found where a vein extends); 2) a loss of tissue flexibility (the flap may feel rigid where a vein lies, and empty where an artery extends); 3) an abnormal color (the flap may look pale where an artery lies, and bruised where a vein extends); and 4) a change of condition at the border of the flap (blisters may occur around the sutures).

Therefore, Taiwan Patent No. 1507172, titled “INFRARED THERMOGRAPHY SYSTEM AND METHOD FOR ANALYZING FACTORS THAT INFLUENCE SURFACE TEMPERATURE OF FREE FLAP”, discloses obtaining the actual surface temperature of a free flap on a living body via an infrared thermographer; obtaining a core temperature of the living body and an ambient temperature corresponding to the environment where the living body is; performing a thermal conduction- and thermal convection-related calculation on the core temperature and the ambient temperature to produce an estimated surface temperature; and according to the closeness between the estimated surface temperature and the actual surface temperature, determining whether factors influencing the surface temperature of the free flap have changed. Any change thus found will be used for compensation and calibration, in order to increase the accuracy of the surface temperature taken by the infrared thermographer of the free flap as a reference indicator.

However, the conventional observation methods are subject to the surroundings and therefore tend to be inaccurate. If an invasive detection method is used instead, a lower level of comfort can be expected during detection.

### BRIEF SUMMARY OF THE INVENTION

To overcome the aforementioned drawbacks of the prior art, the present invention provides a non-invasive method for assessing the survival of a transplanted flap, wherein the method is carried out as follows:

A. A constant current is generated at a fixed frequency and passed through a detection electrode. B. The detection electrode detects a bioelectrical impedance of the skin to which the flap has been transplanted. C. The bioelectrical impedance of the skin with the transplanted flap is compared with a pre-defined threshold value, and the former exceeding the latter is identified as an abnormal condition.

Preferably, step C is performed by subtracting a pre-transplantation bioelectrical impedance of the skin from a post-transplantation bioelectrical impedance of the skin to produce a difference, and the difference exceeding a pre-defined threshold value is identified as the abnormal condition.

Preferably, step C is performed by dividing a post-transplantation bioelectrical impedance of the skin that is detected with a relatively low-frequency current by a post-transplantation bioelectrical impedance of the skin that is detected with a relatively high-frequency current to produce a quotient, and the quotient exceeding a pre-defined threshold value is identified as the abnormal condition.

Preferably, step C is performed by standardizing a bioelectrical impedance of the skin, and either Im1(t)/Im1(0) or Im1(t)/Im2(0) exceeding a pre-defined threshold value is identified as the abnormal condition, wherein: Im1(t) is the ratio of a post-transplantation bioelectrical impedance of the skin that is detected over time with a relatively low-frequency current to a post-transplantation bioelectrical impedance of the skin that is detected over time with a relatively high-frequency current, Im1(0) is the ratio of a pre-transplantation bioelectrical impedance of the skin that is detected with the relatively low-frequency current to a pre-transplantation bioelectrical impedance of the skin that is detected with the relatively high-frequency current, and Im2(0) is the ratio of a bioelectrical impedance of a normal skin that is detected with the relatively low-frequency current to a bioelectrical impedance of the normal skin that is detected with the relatively high-frequency current.

The present invention also provides a non-invasive system for assessing the survival of a transplanted flap by the foregoing method, wherein the non-invasive system includes: a control unit; a variable-frequency current generator circuit electrically connected to the control unit; a pair of detection electrodes electrically connected to the variable-frequency current generator circuit; and a voltage-reading circuit electrically connected to the detection electrodes and the control unit.

The control unit instructs the variable-frequency current generator circuit to generate the constant current. The voltage-reading circuit reads the potential difference between the detection electrodes. Then, the control unit calculates the bioelectrical impedance of the skin with the transplanted flap according to the constant current and the potential difference, and compares the bioelectrical impedance with the pre-defined threshold value.

The non-invasive system of the present invention further includes a control input interface circuit, which is electrically connected to the control unit and through which the desired frequency values can be input.

The non-invasive system of the present invention further includes a wireless transmission circuit electrically connected to the control unit in order to output the data obtained by the control unit.

The non-invasive system of the present invention further includes a display interface circuit electrically connected to the control unit in order to output the data obtained by the control unit.

The foregoing technical features produce the following advantageous effects:

1. The survival of a transplanted flap can be accurately assessed by measuring a bioelectrical impedance of the skin to which the flap has been transplanted, and this approach is less subject to the surroundings than the conventional ones.

2. The non-invasive detection system and method of the present invention feature a high level of comfort during detection and are therefore suitable for long-term monitoring applications.

### BRIEF DESCRIPTION OF THE SEVERAL VIEWS OF THE DRAWINGS

FIG. 1 is a function block diagram of the system of the present invention;

FIG. 2 shows bioelectrical impedance curves plotted against current frequency according to an embodiment of the present invention;

FIG. 3 shows bioelectrical impedance curves plotted for the first determination method of the present invention;

FIG. 4 shows bioelectrical impedance curves plotted for the second determination method of the present invention; and

FIG. 5 shows bioelectrical impedance curves plotted for the third determination method of the present invention.

### DETAILED DESCRIPTION OF THE INVENTION

The present invention incorporates the foregoing technical features into a non-invasive method and system for assessing the survival of a transplanted flap, whose major effects are demonstrated by the following embodiments.

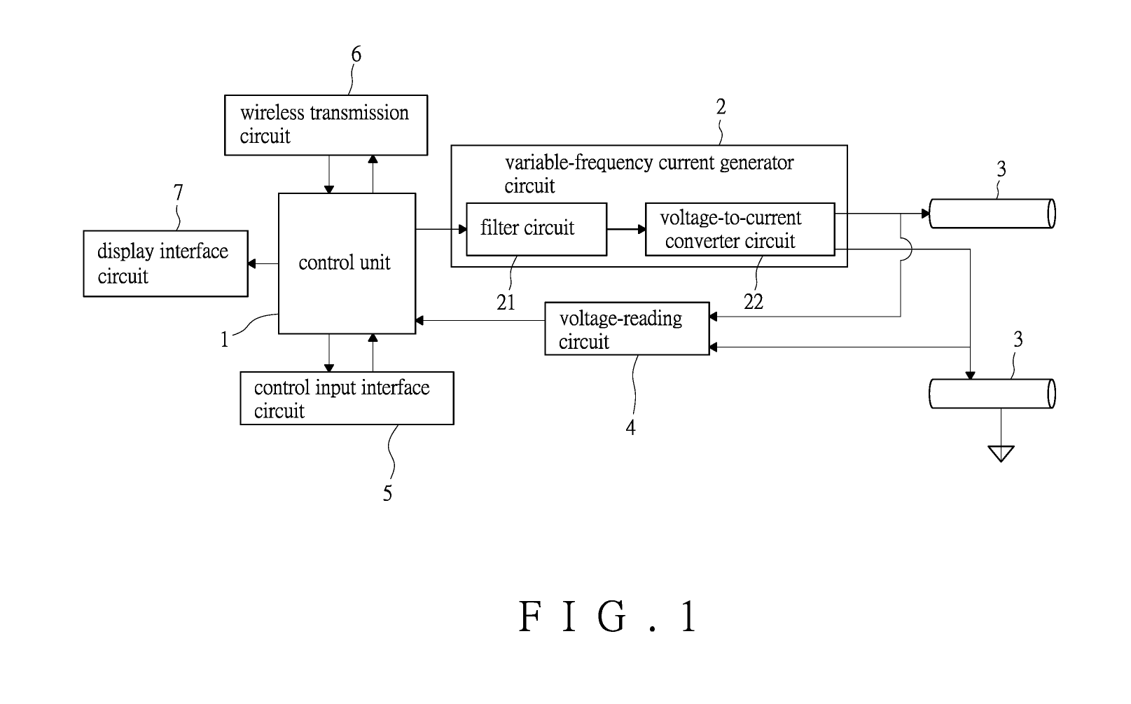

Referring to FIG. 1, the system according to an embodiment of the present invention includes: a control unit 1; a variable-frequency current generator circuit 2 electrically connected to the control unit 1 and including a filter circuit 21 and a voltage-to-current converter circuit 22; a pair of detection electrodes 3 electrically connected to the variable-frequency current generator circuit 2; a voltage-reading circuit 4 electrically connected to the detection electrodes 3 and the control unit 1; a control input interface circuit 5 electrically connected to the control unit 1; a wireless transmission circuit 6 electrically connected to the control unit 1; and a display interface circuit 7 electrically connected to the control unit 1.

In this embodiment, the foregoing system is configured to perform a method including the following steps:

A. The control unit 1 instructs the variable-frequency current generator circuit 2 to generate a constant current at a fixed frequency and pass the constant current through the detection electrodes 3. The desired frequency value can be input through the control input interface circuit 5.

B. The detection electrodes 3 detect a bioelectrical impedance of a skin to which a flap has been transplanted and of a normal skin. More specifically, the voltage-reading circuit 4 reads the potential difference between the detection electrodes 3, in order for the control unit 1 to calculate the bioelectrical impedance of the skin with the transplanted flap or of the normal skin according to the constant current and the potential difference.

C. The bioelectrical impedance of the skin with the transplanted flap is compared with a pre-defined threshold value. When the bioelectrical impedance of the skin with the transplanted flap exceeds the pre-defined threshold value, it is determined that an abnormal condition has occurred. The pre-defined threshold value is determined by a medical professional. For example, the difference in bioelectrical impedance between the skin with the transplanted flap and the normal skin (i.e., the variation of bioelectrical impedance of the skin with the transplanted flap) is calculated. When the ratio of the variation to the bioelectrical impedance of the normal skin exceeds a certain percentage (meaning the variation is significant), it is determined that the skin with the transplanted flap is in an abnormal condition (e.g., necrosis has taken place). The pre-defined threshold value will be dealt with further below in association with different determination methods.

The wireless transmission circuit 6 is configured to transmit the data obtained by the control unit 1 to a backend PC or other processer. The display interface circuit 7 is configured to output the data obtained by the control unit 1.

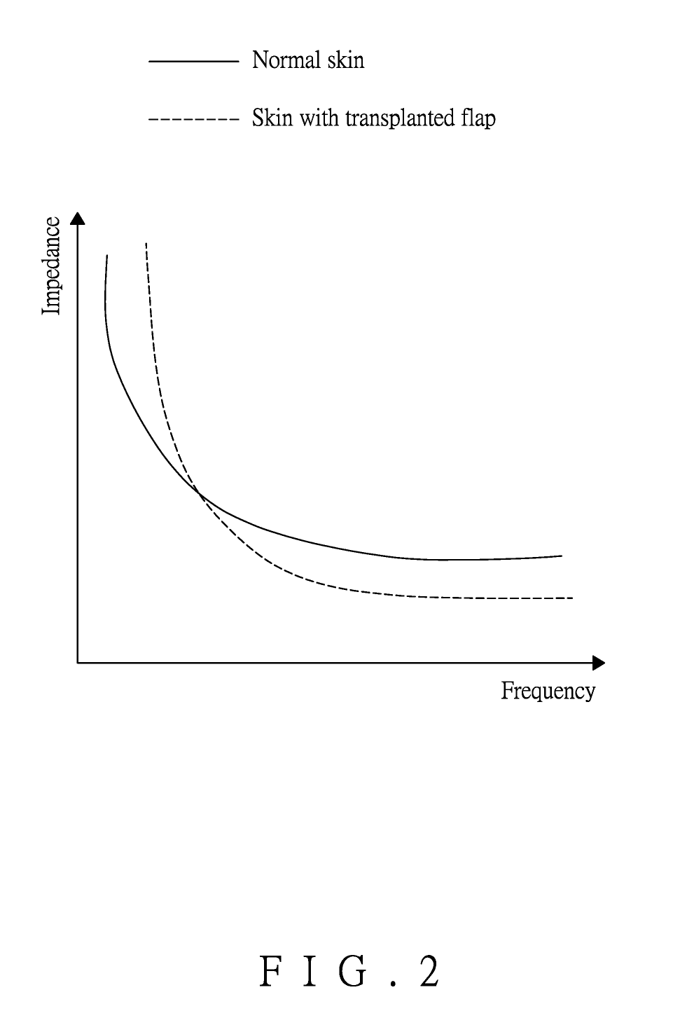

FIG. 2 shows curves that respectively represent the bioelectrical impedance of the skin with the transplanted flap and of the normal skin in relation to the frequency of the detection current. As shown in the drawing, relatively low-frequency currents pass through only a superficial layer of the flap, whereas relatively high-frequency currents pass through deeper layers of the flap. The “relatively low” and “relatively high” frequencies can be determined by a person of ordinary skill in the art based on bioelectrical impedance-related knowledge and therefore should be viewed as specific.

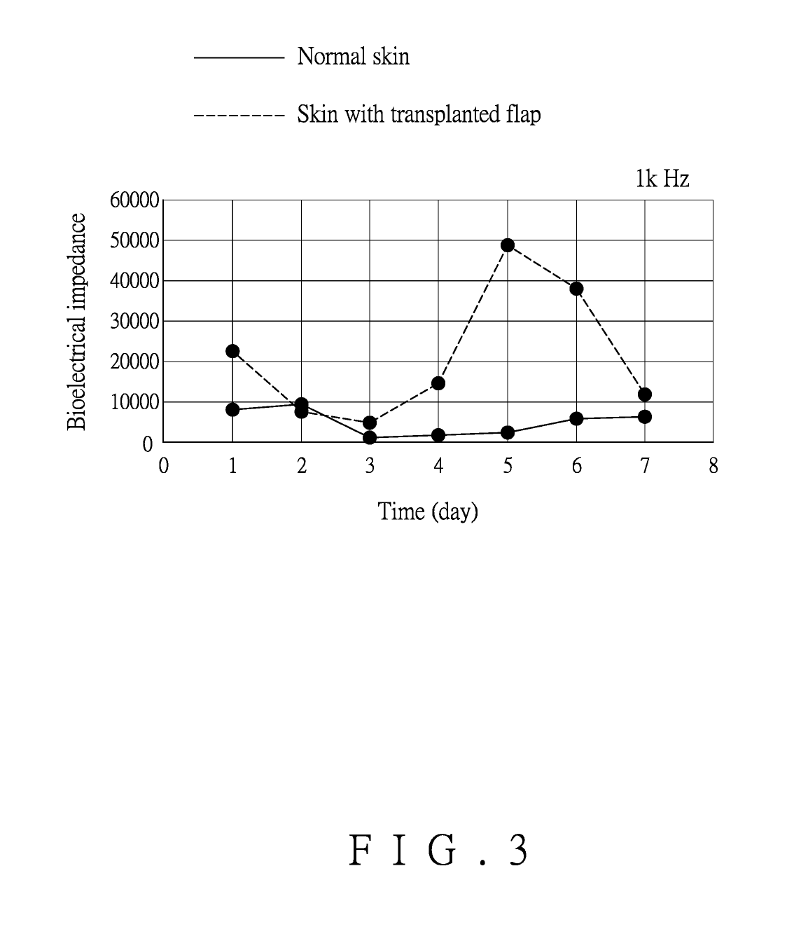

FIG. 3 shows curves that respectively represent the bioelectrical impedance of the skin with the transplanted flap and of the normal skin in relation to time, wherein the frequency of the detection current is 1 KHz. To more accurately determine the survival condition of the skin with the transplanted flap, step C may be carried out by subtracting a pre-transplantation bioelectrical impedance of the skin with the transplanted flap from a post-transplantation bioelectrical impedance of the skin with the transplanted flap to produce a difference, and if the difference exceeds a pre-defined threshold value, determining that the abnormal condition has occurred. Generally, this pre-defined threshold value should be greater than zero in order to reflect whether the post-transplantation bioelectrical impedance of the skin with the transplanted flap is different from the bioelectrical impedance of normal skin. It should be pointed out that, as shown in FIG. 3, the post-transplantation bioelectrical impedance of the skin with the transplanted flap rises in the fourth through the sixth days and falls in the sixth and the seventh days to approach the bioelectrical impedance of the normal skin. One possible explanation for this phenomenon is that edema of a vein in the skin with the transplanted flap causes a rise in bioelectrical impedance, and that the bioelectrical impedance returns to a normal level as soon as the edema is resolved. In practice, therefore, bioelectrical impedance should be measured on a regular basis for at least seven days in order to determine whether a skin with a transplanted flap is in an abnormal condition.

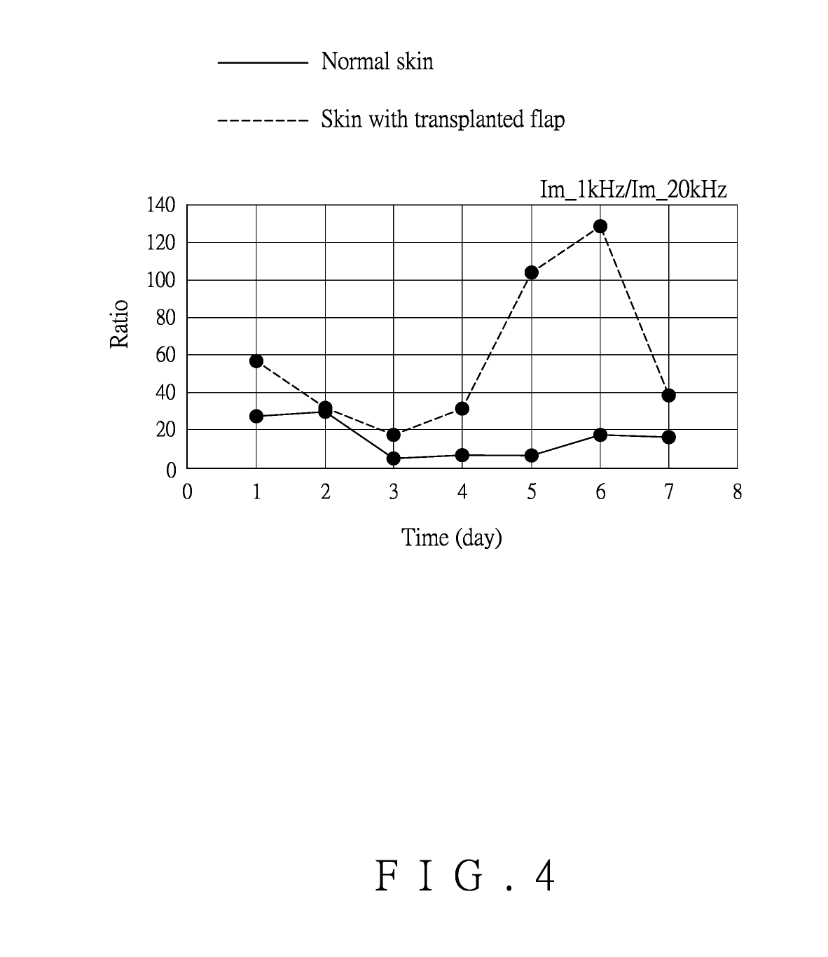

Referring to FIG. 4, step C may alternatively be carried out by dividing a post-transplantation bioelectrical impedance of the skin with the transplanted flap that is detected with a current of a relatively low frequency (e.g., 1 KHz) by a post-transplantation bioelectrical impedance of the skin with the transplanted flap that is detected with a current of a relatively high frequency (e.g., 20 KHz) to produce a quotient, and if the quotient exceeds a pre-defined threshold value, determining that the abnormal condition has occurred. Generally, this pre-defined threshold value should be greater than the pre-transplantation counterpart of the quotient in order to reflect whether the post-transplantation quotient of the skin with the transplanted flap is different from the quotient of normal skin.

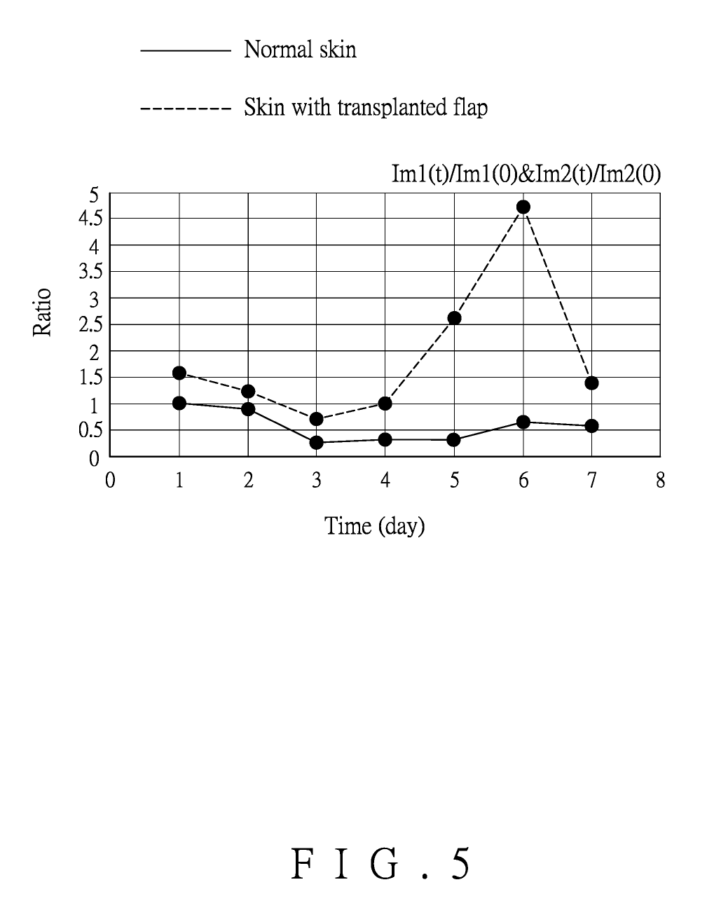

Referring to FIG. 5, step C may also be carried out by standardizing a bioelectrical impedance of the skin with the transplanted flap, and if the ratio Im1(t)/Im1(0) or the ratio Im1(t)/Im2(0) exceeds a pre-defined threshold value, determining that the abnormal condition has occurred, wherein: Im1(t) is the ratio of a post-transplantation bioelectrical impedance of the skin with the transplanted flap that is detected over time with a current of a relatively low frequency to a post-transplantation bioelectrical impedance of the skin with the transplanted flap that is detected over time with a current of a relatively high frequency, Im1(0) is the ratio of a pre-transplantation bioelectrical impedance of the skin with the transplanted flap that is detected with the current of the relatively low frequency to a pre-transplantation bioelectrical impedance of the skin with the transplanted flap that is detected with the current of the relatively high frequency, Im2(0) is the ratio of a bioelectrical impedance of a normal skin that is detected with the current of the relatively low frequency to a bioelectrical impedance of the normal skin that is detected with the current of the relatively high frequency, and generally the pre-defined threshold value should be greater than 1 in order to reflect whether the standardized post-transplantation bioelectrical impedance ratio of the skin with the transplanted flap is different from the standardized bioelectrical impedance ratio of normal skin. As shown in FIG. 5, the standardized bioelectrical impedance ratio of the normal skin is calculated from Im2(t), which is the ratio of a bioelectrical impedance of the normal skin that is detected over time with the current of the relatively low frequency to a bioelectrical impedance of the normal skin that is detected over time with the current of the relatively high frequency.

The embodiments described above should be able to enable a person of ordinary skill in the art to fully understand the operation, use, and effects of the present invention. Those embodiments, however, are but some preferred embodiments of the invention and are not intended to be restrictive of the scope of the invention. All simple, equivalent changes and modifications made according to the appended claims and the disclosure of this specification should fall within the scope of the present invention.

What is claimed is:

1. A non-invasive method for assessing survival of a transplanted flap, comprising the steps of:
A. generating a constant current at a fixed frequency, and passing the constant current through a detection electrode;
B. detecting with the detection electrode a bioelectrical impedance of a skin with a transplanted flap; and
C. comparing the bioelectrical impedance of the skin with the transplanted flap with a pre-defined threshold value, and if the bioelectrical impedance of the skin with the transplanted flap exceeds the pre-defined threshold value, determining that an abnormal condition has occurred.

2. The non-invasive method of claim 1, wherein the step C comprises subtracting a pre-transplantation bioelectrical impedance of the skin with the transplanted flap from a post-transplantation bioelectrical impedance of the skin with the transplanted flap to produce a difference, and if the difference exceeds a pre-defined threshold value, determining that the abnormal condition has occurred.

3. The non-invasive method of claim 1, wherein the step C comprises dividing a post-transplantation bioelectrical impedance of the skin with the transplanted flap that is detected with a current of a relatively low frequency by a post-transplantation bioelectrical impedance of the skin with the transplanted flap that is detected with a current of a relatively high frequency to produce a quotient, and if the quotient exceeds a pre-defined threshold value, determining that the abnormal condition has occurred.

4. The non-invasive method of claim 1, wherein the step C comprises standardizing a bioelectrical impedance of the skin with the transplanted flap, and if Im1(t)/Im1(0) or Im1(t)/Im2(0) is greater than a pre-defined threshold value, determining that the abnormal condition has occurred, wherein Im1(t) is a ratio of a post-transplantation bioelectrical impedance of the skin with the transplanted flap that is detected over time with a current of a relatively low frequency to a post-transplantation bioelectrical impedance of the skin with the transplanted flap that is detected over time with a current of a relatively high frequency, Im1(0) is a ratio of a pre-transplantation bioelectrical impedance of the skin with the transplanted flap that is detected with the current of the relatively low frequency to a pre-transplantation bioelectrical impedance of the skin with the transplanted flap that is detected with the current of the relatively high frequency, and Im2(0) is a ratio of a bioelectrical impedance of a normal skin that is detected with the current of the relatively low frequency to a bioelectrical impedance of the normal skin that is detected with the current of the relatively high frequency.

5. A non-invasive system for assessing survival of a transplanted flap by the non-invasive method of claim 1, comprising:
a control unit;
a variable-frequency current generator circuit electrically connected to the control unit;
a pair of said detection electrodes electrically connected to the variable-frequency current generator circuit; and
a voltage-reading circuit electrically connected to the detection electrodes and the control unit;
wherein the control unit instructs the variable-frequency current generator circuit to generate the fixed current, the voltage-reading circuit reads a potential difference between the detection electrodes, and the control unit calculates the bioelectrical impedance of the skin with the transplanted flap according to the constant current and the potential difference and compares the bioelectrical impedance with the pre-defined threshold value.

6. The non-invasive system of claim 5, further comprising a control input interface circuit electrically connected to the control unit to enable input of a desired frequency value.

7. The non-invasive system of claim 5, further comprising a wireless transmission circuit electrically connected to the control unit to output data obtained by the control unit.

8. The non-invasive system of claim 5, further comprising a display interface circuit electrically connected to the control unit to output data obtained by the control unit.

9. A non-invasive system for assessing survival of a transplanted flap by the non-invasive method of claim 2, comprising:
a control unit;
a variable-frequency current generator circuit electrically connected to the control unit;
a pair of said detection electrodes electrically connected to the variable-frequency current generator circuit; and
a voltage-reading circuit electrically connected to the detection electrodes and the control unit;
wherein the control unit instructs the variable-frequency current generator circuit to generate the fixed current, the voltage-reading circuit reads a potential difference between the detection electrodes, and the control unit calculates the bioelectrical impedance of the skin with the transplanted flap according to the constant current and the potential difference and compares the bioelectrical impedance with the pre-defined threshold value.

10. The non-invasive system of claim 9, further comprising a control input interface circuit electrically connected to the control unit to enable input of a desired frequency value.

11. The non-invasive system of claim 9, further comprising a wireless transmission circuit electrically connected to the control unit to output data obtained by the control unit.

12. The non-invasive system of claim 9, further comprising a display interface circuit electrically connected to the control unit to output data obtained by the control unit.
